(19) United States
(12) Patent Application Publication
(10) Pub. No.: US 2023/0185382 A1
(43) Pub. Date: Jun. 15, 2023

(54) UNIVERSAL SENSOR ALLOWING CONFIGURATION OF INPUTS FOR GATHERING PATIENT FEEDBACK

(71) Applicant: Reciprocal Labs Corporation, Madison, WI (US)

(72) Inventors: Robert Louis BADDELEY, Madison, WI (US); Phuong Linh Thi HOANG, Madison, WI (US)

(21) Appl. No.: 18/005,317

(22) PCT Filed: Jul. 12, 2021
(86) PCT No.: PCT/US2021/041307

§ 371 Date: Jan. 12, 2023

Related U.S. Application Data
(60) Provisional application No. 63/051,139, filed on Jul. 13, 2020.

(51) Int. Cl. G06F 3/02; G16H 20/10; A61M 15/00; G06F 3/04847

(52) U.S. Cl. CPC G06F 3/0202; G16H 20/10; A61M 15/008; G06F 3/04847; A61M 2205/502; A61M 2205/3561

(57) ABSTRACT

A configurable universal sensor directed toward collection of user input health related data is disclosed. The sensor includes a user operated configurable input such as a button. The sensor includes a transceiver operable to communicate with an external device. The sensor includes a controller coupled to the configurable input and the transceiver. The controller collects data from the configurable input and classifies the data according to a configured data type.

### CROSS-REFERENCE TO RELATED APPLICATIONS

This application claims the benefit of, and priority to, U.S. Provisional Patent Application No. 63/051,139, filed Jul. 13, 2020, which is hereby incorporated by reference herein in its entirety.

### TECHNICAL FIELD

The present disclosure relates generally to medication tracking, and more specifically for universal sensor that may be configured for recording relevant medication events such as taking medication.

### BACKGROUND

Currently, many patients with respiratory ailments are provided an inhaler to provide dosages of drugs. For example, an asthma patient may be provided a stimulant to assist mucus and reducing inflammation in clearing up breathing passages. Thus, when the patient experiences asthma exacerbations, the patient can put the inhaler in front their mouth and activate the inhaler spray, delivering a dose of the drug into the lungs in order to relieve the symptom.

It is desirable for providers to be able to determine when a patient administers the medication from the inhaler For example, such data may allow a health care provider to determine whether the medication is effective to treat the respiratory ailment. Further, the data may be used to support refills by tracking the doses that remain available.

Currently, certain advanced inhalers may provide some electronic indicators that the pump has been activated and thus may report via electronic communication when an inhaler has been activated to interested parties such as physicians or payors. Another solution may be a sensor that may be attached to certain models of inhalers to collect activation data. One example of such a device is the Sensor Model 2016-M, Sensor for MDI manufactured by Propeller Health of Madison, Wis.

Thus, the most advanced current inhaler systems allow physicians and/or payors to determine that the pump on an inhaled drug has been activated and therefore track the medication. However, there may be certain inhalers that do not have built-in sensors or are compatible with attachable sensors. Further, other medication in traditional pill forms cannot be tracked. Another solution may be the provision of an application on a personal mobile device such as a smartphone. When a patient uses an inhaler, the patient may record the dose on the application, and thus provide the activation data.

However, for many patients, it is inconvenient to pull out their mobile device, open the application, and select the correct interfaces to enter data. Another issue is that a patient may forget to enter the data when taking medication or may not have access to their mobile device.

There is a need for a device that may be configured to allow efficient and accessible recording of data from a patient in relation to different non-inhaler medications. There is a further need for a device that may be configured to record triggering conditions and events related to medication taken by a patient. There is also a need for a device that may be configured to record different types of data inputs from a patient.

### SUMMARY

One disclosed example is a configurable sensor directed toward collection of user input data. The sensor includes a user operated configurable input. The sensor includes a transceiver operable to communicate with an external device. A controller is coupled to the configurable input and the transceiver. The controller collects data from the configurable input and classify the data according to a configured data type.

A further implementation of the example sensor is where the configured data type is one of user medication, user dose, user emotion, user activity, or user trigger. Another implementation is where the transceiver is a blue tooth transceiver. Another implementation is where the sensor includes a housing holding the transceiver and controller. The housing has a base exterior surface and an opposite open aperture. The user operated configurable input is a push-button extending through the open aperture. Another implementation is where the base exterior surface includes an adhesive for attachment to a flat surface. Another implementation is where the base exterior surface includes a loop structure for attaching a lanyard or strap. Another implementation is where the sensor includes a faceplate having button areas, the configurable input being one of the button areas. Another implementation is where the faceplate is a display screen. Another implementation is where the sensor includes a decal overlaying the faceplate. The decal includes identification information for the button areas associated with the configured data type. Another implementation is where the decal includes icons on each of the button areas. The icons correspond to the configured data type. Another implementation is where the configured data type includes related data inputs. Each data input corresponds to each one of the button areas. Another implementation is where each of the button areas includes a tactile surface to identify the button area. Another implementation is where the external device is a server including a health analysis engine. The health analysis engine receives the collected data corresponding to the configured data type; analyzes the collected data; and outputs a predicted health condition relating to the user. Another implementation is where the external device is a mobile device. The controller sends the collected data via the transceiver to the mobile device when the mobile device is within transmission range of the configurable sensor. Another implementation is where the mobile device is paired with the configurable sensor for collected the collected data. Another implementation is where the mobile device is paired with a second configurable sensor. The second configurable sensor has a user operated configurable input and a transceiver operable to communicate with an external device. A controller is coupled to the configurable input and the transceiver. The controller collects data from the configurable input and classifies the data according to a configured data type. The controller sends the collected data via the transceiver to the mobile device when the mobile device is within transmission range of the second configurable sensor. Another implementation is where the second configurable sensor is configured to collect a different type of data from the data type configured in the first configurable sensor. Another implementation is where the mobile device includes a configuration interface that configures the configured data type of the configurable sensor corresponding to the configurable input. The mobile device sends a configuration signal to the transceiver to configure the configurable input and the configurable data type. Another implementation is where the user input is an actuator device coupled remotely from the transceiver and the controller. Another implementation is where the sensor further includes another user operated input that activates a non-configurable function.

Another disclosed example is a data collection system having a first configurable sensor directed toward collection of user input data. The sensor has a user operated configurable input and a transceiver operable to communicate with an external device. A controller is coupled to the configurable input and the transceiver, the controller operable to collect data from the configurable input and classify the data according to a configured data type. A data server receives the collected classified data from the transceiver. An analysis module determines a health condition of the user based on the collected classified data.

A further implementation of the example system is where the configured data type is one of user medication, user dose, user emotion, user activity, or user trigger. Another implementation is where the transceiver is a blue tooth transceiver. Another implementation is where the sensor includes a housing holding the transceiver and controller. The housing has a base exterior surface and an opposite open aperture. The user operated configurable input is a push-button extending through the open aperture. Another implementation is where the base exterior surface includes an adhesive for attachment to a flat surface. Another implementation is where the base exterior surface includes a loop structure for attaching a lanyard or strap. Another implementation is where the sensor includes a faceplate having button areas. The configurable input is one of the button areas. Another implementation is where the faceplate is a display screen. Another implementation is where the sensor includes a decal overlaying the faceplate. The decal includes identification information for the button areas associated with the configured data type. Another implementation is where the decal includes icons on each of the button areas. The icons correspond to the configured data type. Another implementation is where the configured data type includes related data inputs. Each data input corresponds to each one of the button areas. Another implementation is where each of the button areas includes a tactile surface to identify the button area. Another implementation is where the external device is a mobile device. The controller sends the collected data via the transceiver to the mobile device when the mobile device is within transmission range of the configurable sensor. Another implementation is where the mobile device is paired with the configurable sensor for collecting the collected data. Another implementation is where the mobile device is paired with a second configurable sensor. The second configurable sensor includes a user operated configurable input; a transceiver operable to communicate with an external device; and a controller coupled to the configurable input and the transceiver. The controller is operable to collect data from the configurable input and classify the data according to a configured data type. The controller sends the collected data via the transceiver to the mobile device when the mobile device is within transmission range of the second configurable sensor. Another implementation is where the second configurable sensor is configured to collect a different type of data from the data type configured in the first configurable sensor. Another implementation is where the mobile device includes a configuration interface operable to configure the configured data type of the configurable sensor corresponding to the configurable input. The mobile device sends a configuration signal to the transceiver to configure the configurable input and the configurable data type. Another implementation is where the user input is an actuator device coupled remotely from the transceiver and the controller.

Another disclosed example is a method of collecting user input data from a universal sensor. A user operated configurable input on the universal sensor is configured according to a configured data type. An input is received on the user operated configurable input. The data from the configurable input is collected via a controller. The data is classified according to the configured data type. The classified data is transmitted via a transceiver to an external device.

A further implementation of the example method is where the configured data type is one of user medication, user dose, user emotion, user activity, or user trigger. Another implementation is where the transceiver is a blue tooth transceiver. Another implementation is where the sensor includes a housing holding the transceiver and controller. The housing has a base exterior surface and an opposite open aperture. The user operated configurable input is a push-button extending through the open aperture. Another implementation is where the base exterior surface includes an adhesive for attachment to a flat surface. Another implementation is where the base exterior surface includes a loop structure for attaching a lanyard or strap. Another implementation is where the universal sensor further includes a faceplate having button areas. The configurable input is one of the button areas. Another implementation is where the faceplate is a display screen. Another implementation is where the universal sensor includes a decal overlaying the faceplate. The decal includes identification information for the button areas associated with the configured data type. Another implementation is where the decal includes icons on each of the button areas, where the icons correspond to the configured data type. Another implementation is where the configured data type includes related data inputs. Each data input corresponds to each one of the plurality of button areas. Another implementation is where each of the button areas includes a tactile surface to identify the button area. Another implementation is where the external device is a mobile device. The controller sends the collected data via the transceiver to the mobile device when the mobile device is within transmission range of the configurable sensor. Another implementation is where the mobile device is paired with the configurable sensor for collected the collected data. Another implementation is where the mobile device is paired with a second configurable sensor. The second configurable sensor includes a user operated configurable input; a transceiver operable to communicate with an external device; and a controller coupled to the configurable input and the transceiver. Data is collected from the configurable input and classified according to a configured data type. The collected data is sent via the transceiver to the mobile device when the mobile device is within transmission range of the second configurable sensor. Another implementation is where the second configurable sensor is configured to collect a different type of data from the data type configured in the first configurable sensor. Another implementation is where the configured data type of the configurable sensor corresponding to the configurable input is configured via a configuration interface of the mobile device. A configuration signal is sent to the transceiver to configure the configurable input and the configurable data type.

Another implementation is where the user input is an actuator device coupled remotely from the transceiver and the controller. Another implementation is where the method includes activating a function in response to a user operated non-configurable input.

Another disclosed example is a computer program product comprising instructions which, when executed by a computer, cause the computer to carry out the above methods. Another implementation is where the computer program product is a non-transitory computer readable medium.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1A is a perspective view of an example universal sensor device;

FIG. 1B is an exploded view of the circuit board components of the example universal sensor device in FIG. 1A;

FIG. 1C is another exploded view of an underlying contact pad of the example universal sensor device in FIG. 1A;

FIG. 2 is a block diagram of a data collection system that allows collection of user input data from the universal sensor device in FIG. 1A;

FIGS. 3A-3C are example configurations of the universal sensor device in FIG. 1A;

FIG. 3D shows several examples of the incorporation of the universal sensor device with other sensor actuation devices;

FIG. 4A-4C are alternate example button configurations of the example universal sensor device in FIG. 1A;

FIGS. 5A-5D are example interfaces on an application for a mobile device for pairing and configuring the universal sensor device in FIG. 1A;

FIGS. 6A-6D are example interfaces of the application on the mobile device for configuring buttons on the universal sensor device;

FIG. 7 is a flow diagram of the routine for a mobile device receiving collected data from the example universal sensor in FIG. 1A;

FIG. 8 is a block diagram of a health care system that supports the data collected by the universal sensor device in FIG. 1A;

FIG. 9A is a perspective view of another example universal sensor device;

FIG. 9B is an exploded view of the components of the example universal sensor device in FIG. 9A;

FIG. 9C shows the flexible circuit board of the universal sensor device in FIG. 9A; and

FIG. 10 is a perspective view of an alternate base component for the example universal sensor device in FIG. 9A.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a universal sensor that may be configured to record patient inputs related to the administration of medication. The universal sensor has one or more user configurable inputs such as buttons. Each of the inputs has a customizable configured meaning in relation to data input by the user. Once the buttons are configured, when a patient presses a button, the universal sensor records data signifying an event that is classified or defined by the configuration. For example, the buttons may be programmed to record a triggering event, an activity, a use of medication, or subjective patient feedback. The internal electronics and enclosure remain the same for each universal sensor. However, the meaning for the buttons are associated with different faceplates that may be attached over the configured buttons. Data is then collected and classified according to the configuration of the buttons.

The data is collected by the universal sensor and wirelessly communicated to a remote server either directly or through a mobile device such as a smartphone. The data may be analyzed to provide diagnostic information regarding the effectiveness of the medication and other useful information relating to the user.

An initial setup application on a mobile computing device maps each button to a particular input data type via a user application. The user then puts the universal sensor in relevant physical locations for recording usage and other data in relation to medication. For example, a universal sensor configured to record patient feeling might be put next to the bed of the patient. A universal sensor configured to record medication might be attached to a nebulizer or a drug cabinet. A universal sensor configured to record activities may be put on a door frame of a room where a patient might start their activities.

In operation, the user presses the appropriate configured button on the universal sensor whenever an event, such as taking medication, occurs. The collected data is synchronized through a mobile device or a network hub. Depending on the data metric collected, data is passed appropriately to an analysis engine.

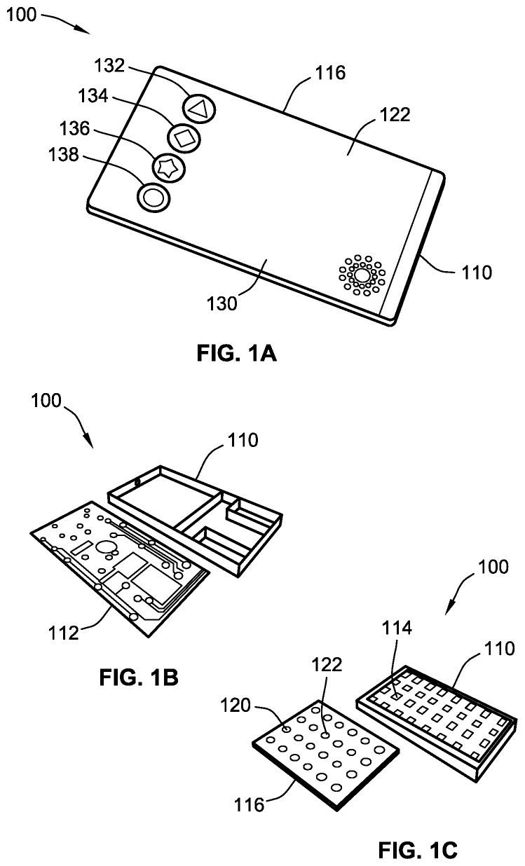

FIG. 1A is a perspective view of an example universal sensor device 100. FIGS. 1B and 1C are exploded views of the components of the example universal sensor device 100. The universal sensor device 100 includes an enclosure 110. The enclosure 110 is constructed from a molded plastic material. The enclosure 110 includes compartments for components such as a battery and supports a printed circuit board 112. The enclosure 110 has an exterior back surface that may include an attachment device such as a clip, or an adhesive may be applied to allow the universal sensor device 100 to be attached to a surface near an area where a user is likely to perform an activity or experience a condition to allow the user to easily enter the relevant data.

A keypad 114 is positioned over the printed circuit board 112. The keypad 114 includes multiple actuators that provide electrical signals to the printed circuit board 112 when pressure is applied to the different actuators. The printed circuit board 112 includes circuits that provide input signals when pressure is detected by the actuators of the keypad 114. A faceplate 116 is attached to the enclosure 110 to enclose the printed circuit board 112. The faceplate 116 has an undersurface 118 that includes contact pads 120 in contact with the keypad 114 to apply pressure to the actuators on the keypad 114. In this example, there are twenty four contact pads 120 and corresponding actuators on the keypad 114. Any number of the twenty four contact pads 120 may be activated for different functions as will be explained herein.

The faceplate 116 includes an opposite top surface 122 that is viewable by a user. In this example, the top surface 122 of the faceplate 116 includes a display area 130 and four configurable button areas 132, 134, 136, and 138. Each of the button areas 132, 134, 136, and 138 has a uniquely shaped icon associated with the button area. The icons may also have a unique color or pattern to further distinguish each of the button areas 132, 134, 136, and 138. The button areas may also be formed of a different tactile pattern to assist in distinguishing the buttons. In this example, the contact pads under the button areas 132, 134, 136, and 138 are active to detect pressure applied by a user on the area. The remaining areas that have an underlying contact pad 120 are deactivated in this example.

As will be explained, the universal sensor device 100 may be paired with a portable mobile device such as a smart phone to allow a user to configure the button areas 132, 134, 136, and 138. The display area 130 is generally smooth and a decal showing the configuration of the button areas may be applied to the display area 130. In some examples, multiple smaller decals may be provided and applied next to the button areas based on the configuration of the button areas. Alternatively, the display area may be an actual electronic display such as an LCD or OLED screen that may display graphics indicating the configuration of the button areas 132, 134, 136, and 138. Once the pairing is established, the data collected by the universal sensor device 100 may be transmitted to the portable mobile device, whenever the portable mobile device is within transmission range of the universal sensor device 100.

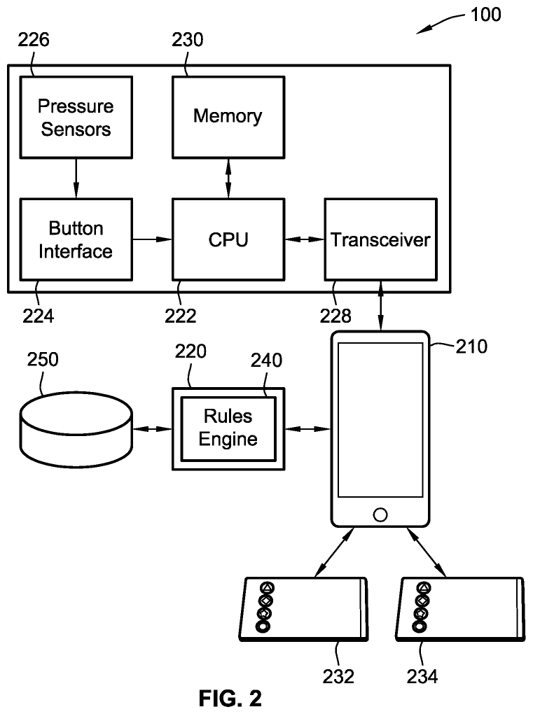

FIG. 2 is a block diagram of the components of a data collection system 200 that includes the universal sensor device 100. The system 200 includes a portable/mobile computing device such as a smartphone 210 and a server 220. The universal device 100 is in wireless communication with the smartphone 210. The wireless communication in this example may be a Bluetooth low energy (BLE) signal having a transmission range of 100 meters. The smartphone 210 may communicate via a network 212 such as the Internet to the server 220. The smartphone 210 executes an application that allows the user to configure the button areas on the universal device 100. The application may also have the capability to collect the data from the configured universal device 100, conduct some analysis of the data, and transmit the data. Thus, data collected by the universal device 100 is sent to the application of the smartphone 210 for transmission to the server 220 in this example A health data analysis engine 240 may be executed on the server 220 to analyze the data.

The universal sensor 100 includes a central processor or controller 222, a button interface 224, a set of pressure transducers 226, and a transceiver 228. The controller 222 may be coupled to a memory device 230. Such a memory may be a volatile type computer memory, including for example RAM, DRAM, or SRAM, may be used. In such instances, the universal sensor 100 may continually transmit data to a computing device such as the smartphone 210 or the server 220 external to the universal sensor 100. In other embodiments non-volatile memory formats may be used, including for example ROM, EEPROM, flash memory, ferroelectric RAM (F-RAM), optical and magnetic computer memory storage devices, and others. The memory 230 may be used to store data collected by the universal sensor 100 as well as configuration information for the buttons on the universal sensor 100.

The faceplate 116 in FIG. 1A is in communication with the button interface 224 that translates signals from the pressure transducers 226 in contact with the keypad 114 to receive inputs that are defined according to the configuration of the button areas of the universal device 100. The transceiver 228 allows exchange of input data between the universal device 100 and the smartphone 210. The transceiver 228 may also receive control data from the smartphone 210 for purposes of configuring the button areas in FIG. 1A-1B. The universal device 100 is configured to collect data input from a user such as when a medication dose is administered by a user or when a triggering event occurs. In this example, the wireless communication may be any suitable protocol including Bluetooth Low Energy. Wi-Fi (IEEE 802.11), Bluetooth®, other radio frequencies, Infra-Red (IR), GSM, CDMA, GPRS, 3G, 4G, W-CDMA, EDGE or DCDMA200 or similar protocols. Although, the example universal sensor 100 communicates and is paired with the smartphone 210, the transceiver 228 may allow direct communication of data to a network for the remote server 220. Further, the configuration may be made from other remote devices in communication with the universal sensor 100.

In this example, the transceiver 228 sends a periodic advertising signal. If the smartphone 210 is within transmission range, the smartphone 210 will receive the periodic advertising signal from the transceiver. When new data has been collected by the universal sensor 100, the advertising signal will include a flag indicating that new data is available. When the smartphone 210 is in communications range, the new data will be transmitted from the universal sensor 100. Once an acknowledgement signal of data receipt is received, the transceiver 228 continues to send the normal advertising signal.

The remote server 220 may be an application server that includes the health data analysis engine 240. The server may include access to a database 250 that includes big data from other users with similar treatment or taking similar medications. Such data may be used to refine the baseline comparison to determine whether a drug is having the desired effect. The database 250 may also include other relevant data relating to the user to assist in evaluation of the condition of the patient.

There may be common data entries in a predetermined template which are device assigned, where the mapping/configuration of the buttons and input data represented by the buttons are pre-assigned. For example, there may be four general configurations (medication, trigger, mood, and activity) for the button areas of the universal sensor 100 provided, each for collection of different types of patient input data. Of course, other configurations may be designed and applied to the universal sensor 100 to collect other types of patient input data.

The medication configuration can collect adherence data by recording a patient input for each administration of a medication. For example, medication use is associated with adherence and thus adherence data in the form of use data can be used to analyze medication use. A trigger data configuration may be used to collect data when a user encounters a particular trigger, such as pollen. Trigger presence is associated with user triggers and exacerbation likelihood in relation to a respiratory ailment. A mood configuration may collect data relating to the mood of the user that may be defined by labels such as rest, tiredness, or exhaustion. Mood is associated with triggers and exacerbation likelihood for the user. An activity configuration may collect data related to user activity. Thus, a user may input the type of activity they are engaged in. As will be explained, the patient input data may be used for a variety of purposes such as evaluating the effect of medication or determining triggering events for exacerbations.

As will be explained below, the data collected from the universal sensor 100 may be used for a variety of purposes based on the configuration of the buttons. These purposes may include determining adherence of the patient in taking medication, the effectiveness of a medication, or the need for medication or other treatment. Thus, in this example, the data input by the patient is provided in a data package sent by the universal sensor 100 that includes a predetermined number of data points over a period of time. Other universal sensors identical to the universal sensor 100 such as universal sensors 232 and 234 may be paired with the smartphone 210. The example other universal sensors 232 and 234 may be configured to collect different types of data. The collected data is stored and collected by the smartphone 210 when the universal sensors 232 and 234 are within transmission range. Thus, the universal sensor 100 may be configured to collect medication data and be located in a medicine cabinet. The universal sensor 232 may be configured for collecting activity data and be located near the front door of the home of the patient. Thus, as the user carries the smartphone 210 and takes medication, the smartphone 210 can collect data from the universal sensor 100. If the user exits through the front door, when the smartphone 210 is in proximity to the universal sensor 232, it may collect the data related to activities.

In this example, there may be multiple options for associating an action to a meaning. For example, a user may assign a meaning via an application executed by the smartphone 210 and thereby configure an appropriate button. In this example, the user may write or put a decal next to the appropriate button and then creates a mapping using the application on the smartphone 210. After the mapping and assignment, the universal sensor 100 may be deployed for use.

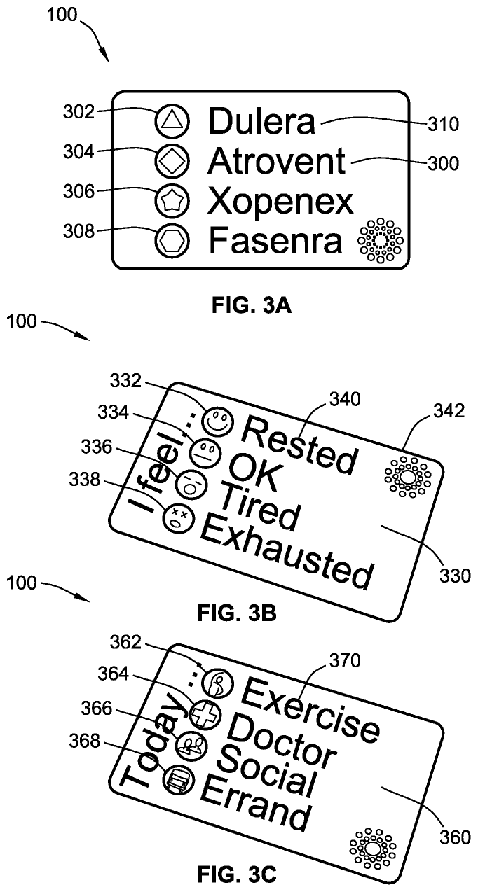

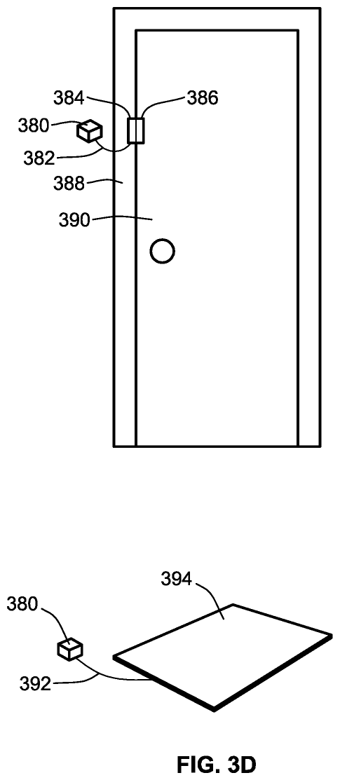

In this example, a template with pre-determined configurations for each of the button areas 132, 134, 136, and 138 as shown in FIG. 1A may be applied after the configurations are established. The templates may include explanatory text and are attached to the top surface of the faceplate 116 of the universal device 100 with adhesive. Alternatively, the templates may be decals with an adhesive backing that can be applied to the faceplate 116. FIG. 3A shows one example of a template 300 related to medication data applied to the surface of the faceplate 116. The template 300 labels a Dulera button 302, an Atrovent button 304, a Xopenex button 306, and a Fasenra button 308. The template 300 also has an area of explanatory labels 310 with the respective medication information in proximity to the buttons 302, 304, 306, and 308. For example, when a user takes Dulera medication, the user will press the button 302 and the universal device 100 will record that the user has taken the Dulera at the time the button 302 is pressed. Each of the buttons 302, 304, 306, and 308 are also provided with graphics that are associated with the medication to assist the user in pressing the correct button. The button areas may also be layered with different tactile patterns to further differentiate the button areas.

FIG. 3B shows another template 330 with predetermined configurations relating to user mood for the button areas 132, 134, 136, and 138 in FIG. 1A. The template 330 labels a restful button 332, an OK button 334, a tired button 336, and an exhausted button 338. The template 330 also has an area of explanatory labels 340 in proximity to the buttons 332, 334, 336, and 338. For example, when a user may be prompted to chart their mood by the application, the user will press the appropriate button 332, 334, 336, or 338 that reflects their current mood. The current mood of the user is then recorded by the universal sensor 100. Each of the buttons 332, 334, 336, and 338 are also provided with face expression graphics that are associated with the corresponding mood to assist the user in pressing the correct button. In this example, the user mood data collected by the configured universal sensor device 100 in FIG. 3B may be used to evaluate the effectiveness of a therapy device such as a continuous positive airway pressure (CPAP) device on a user. Another example may be how a user feels after using a portable oxygen concentrator.

FIG. 3C shows another template 360 with predetermined configurations relating to user activity for the button areas 132, 134, 136, and 138 in FIG. 1A. The template 360 labels an exercise button 362, a doctor button 364, a social button 366, and an errand button 368. The template 360 also has an explanatory labels area 370 with labels in proximity to the buttons 362, 364, 366, and 368. The template 360 allows the universal sensor device 100 to collect user input data related to user activity. For example, when a user exercises, the user will press the button 362 and the universal device 100 will record that exercise has been performed. Each of the buttons 362, 364, 366, and 368 are also provided with graphics that are associated with the activity to assist the user in pressing the correct button. The recording of different activities by the universal sensor 100 configured in FIG. 3C may be used to determine whether certain activities trigger exacerbations that require either medication or use of therapy devices. Alternatively, other templates may collect trigger or symptom data that may also be used to analyze either the occurrence of exacerbations or the prediction of exacerbations. Other types of data may be used to track non-respiratory-health related behavior. For example, other medication use, eating habits or television/media consumption may be tracked. Such data may be used for identifying patterns and links between conditions or behaviors and outcomes like exacerbations. For example, a different medication may have side effects that may be reflected in the collected data. Similarly, conditions such as asthma may be related to a food allergy as may be determined from the collected data.

In the firmware executed by the controller 222, the template that is used on the faceplate 116 is associated with an identifier so that the universal sensor 100 can report not just what buttons were pressed but also what type of faceplate template it has so the buttons have meaning. In the case of pre-determined templates, the user may enter the type of template into an application executed by the smartphone 210. Alternatively, the camera of the smartphone 210 may capture an image of the template and the application may automatically configure the universal sensor 100 for the type of data that is collected. Once data is entered by a user pressing the buttons on the faceplate 116, the collected data is classified by the controller 222 according to the configuration.

The concepts explained here may be applied to other physical configurations for a universal sensor. FIG. 3D shows a universal sensor 380 with electronic components similar to the universal sensor 100 and therefore provides configured input data to an external device such as the smartphone 210 in FIG. 2. In this example, the universal sensor 380 is configured for better accessibility and actuation. The entire universal sensor 380 functions as a single data collection button that is configured to collect certain data. The sensor 380 may either have a larger activation area than the buttons on the universal sensor 100 or be attached to another type of actuator such as a motion sensor, foot pedal, a magnetic switch, or pressure pad for better accessibility and ease of use by a patient. Generally, such an accessibility system includes a universal sensor, a connector and cable leading from the sensor, and a switch or actuator device that is actuated by some means.

FIG. 3D shows several examples of the incorporation of the universal sensor 380 with other sensor actuation devices. In this example, a cable 382 may be attached to a magnetic switch 384 mounted on a door frame 386. A magnetic panel 388 is attached to a door 390. The door 390 thus is employed as a data input device. When a user desires to input data, the user may open the door 390 thereby breaking the magnetic field to the magnetic switch 384 and sending the signal to the sensor 380. Other actuators may be used. For example, the universal sensor 380 may be attached via a cable 392 to a pressure pad 394. When a user desired to input data, the user may step on the pressure pad 394 and thereby send a data signal to the sensor 380. Alternatively, the actuators may be in wireless communication with the sensor 380.

The employment of other actuators allows the action to input data not being not limited to the user pressing a button on the universal sensor itself The other actuators may allow other ways of making contact, including other types of sensors, like light or water or mechanisms that are activated by shaking or tilting, twisting, bopping, squeezing, pulling to indicate a binary input.

The configuration of the buttons on the universal device 100 is performed by an application that runs on a mobile device such as the smartphone 210. The application has the functions of tracking and collecting data in the context of how the universal device 100 is configured. For example, when the medication configuration and template 300 in FIG. 3A is used, when different medications are taken by a user, the universal sensor 100 collects data relating to when the medications are taken. The collected data is then communicated to external devices such as the server 220 through the smartphone 210 that are within transmission range of the universal sensor 100.

There is no limit to the number of buttons that may be employed on the example universal sensor 100. In the example universal sensor 100 in FIG. 1A-1B, only four buttons are used, but the system could easily support any number of buttons. For example, the keypad 114 in FIG. 1B may support up to 24 separate button areas, although only four are used. Further, the arrangement of the buttons on the faceplate 116 may also be changed by activating different ones of the 24 separate button areas. Greater or fewer than 24 button areas in different physical arrangements may also be provided.

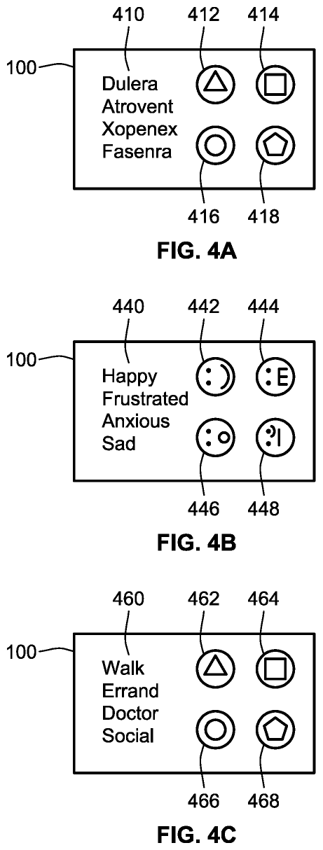

FIGS. 4A-4C show other templates for a different definition of button areas from the 24 buttons that allows for a different button area arrangement on the universal sensor 100 than those arrangements shown in FIGS. 3A-3C. FIG. 4A shows a template 410 that is configured for collected medication data. The template 410 includes four buttons 412, 414, 416, and 418. As may be seen in comparison with FIG. 1A, the location of the buttons 412, 414, 416, and 418 differ from the location of the buttons 132, 134, 136, and 138. In this example, the buttons 412, 414, 416, and 418 use different actuators on the keypad 114. In this example, the template 410 has been configured for collecting medication use data. Thus, the buttons 412, 414, 416, and 418 represent uses of example medications such as Dulera, Atrovent, Xopenex, and Fasenra respectfully. Thus, when a user takes one of the four medications, the user will press the appropriate button 412, 414, 416, and 418. Each of the buttons 412, 414, 416, and 418 has a distinct graphic to assist the user in selecting the correct button corresponding to the respective medication.

FIG. 4B shows an alternate template 440 for the universal sensor 100 that is preconfigured for collecting emotion data. The template 440 includes four buttons 442, 444, 446, and 448, that represent example emotion such as happy, frustrated, anxious, and sad. In this example, the universal sensor 400 allows a user to enter their emotion by pressing the respective button. Each of the buttons 442, 444, 446, and 448 has a graphic relating to the emotion to assist the user in selecting the correct button.

FIG. 4C shows another alternate template 460 for the universal sensor that is configured for collecting activity data. The template 460 includes four buttons 462, 464, 466, and 468, that represent example activities such as walking, running an errand, going to the doctor, or social errand respectively. Each of the buttons 462, 464, 466, and 468 has a distinct graphic to assist the user in selecting the correct button.

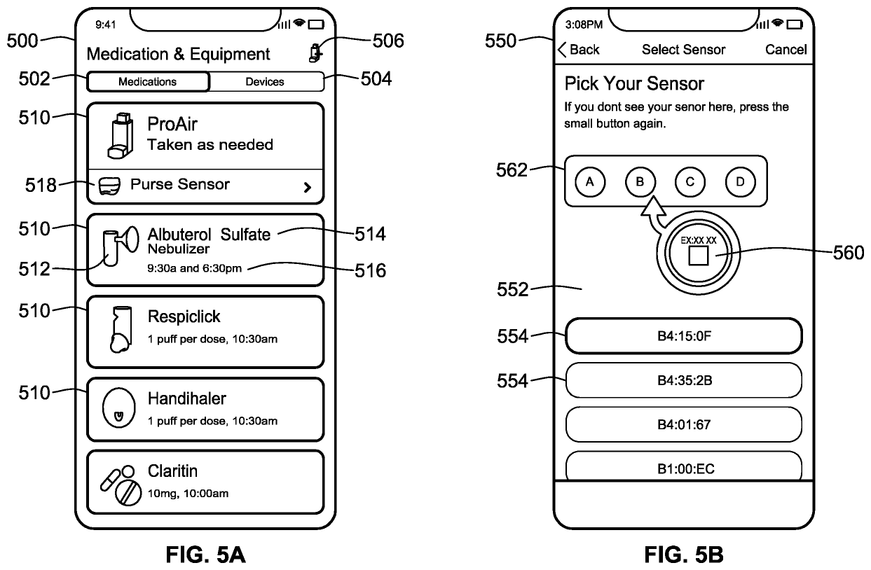

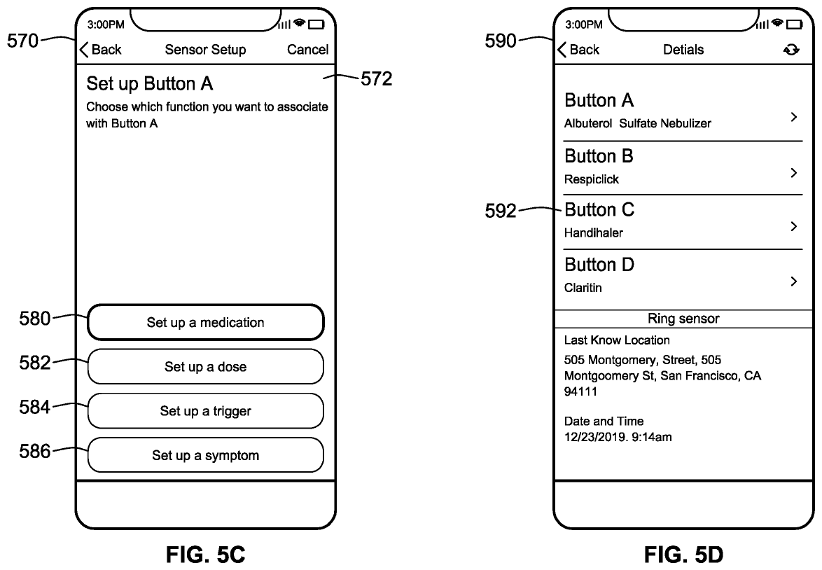

As explained above, the application on the smartphone 210 may be executed by the user to configure the universal sensor 100 to label the configured data for an appropriate template. FIG. 5A shows an example main interface 500 generated by the application that displays a selection between a medications selection 502 and a devices selection 504. The devices selection 504 when selected, shows an interface displaying all of the universal sensors that are paired with the smartphone 210. The application may also display other types of specific sensors that have pairing capabilities and transmit sensed data to the smartphone 210. Thus, when the smartphone 210 is in transmission range of any of the paired universal sensors, the universal sensors transmit any collected data to the application. An add icon 506 is displayed to allow a user to add an additional sensor or medication to be tracked by the application.

The interface 500 is shown when the medications selection 502 is selected. The interface 500 thus shows a listing of medications 510 currently prescribed or available to the user. Each of the listings 510 include a graphic 512 showing the dispensing device or drug, an identifying label 514, and the dosage and time 516. Alternatively, the listing 510 may include a sensor type tab 518 that indicates that the specific sensor or button on the universal sensor 100 is paired to a particular medication. Thus, when the medication in the listing is taken, the user activates the specific sensor or the button on the universal sensor to record the usage.

When the add icon 506 is selected, a menu is displayed that includes an option to add medication and an option to add a new sensor device. If the add a new sensor device option is selected, the new sensor such as the universal sensor 100 must be paired with the smartphone 210. The transmitter on the universal sensor 100 will emit a Bluetooth signal and the application causes the smartphone 210 to recognize the signal and pair the devices. Once the universal sensor 100 is found, it will be listed in a sensor configuration interface 550 as shown in FIG. 5B. The interface 550 includes a list of recognized sensors 552. Each of the recognized sensors in the list 552 has a corresponding selection area 554 that includes a serial number to identify the sensor. Once the sensor is recognized, the smartphone 210 will acknowledge advertising signals received from such sensors and then receive any data collected by the sensor.

Once the sensor is recognized by the application, it may be selected via the corresponding selection area 554. A circle icon 560 is displayed with the serial number of the selected sensor, and a button selection field 562 is displayed to allow each button to be configured. In this example, the button selection field 562 is shown to allow a user to select one of the four buttons on the universal sensor 100 in FIG. 1A. If there are more or less buttons, more or less options may be shown on the button selection field 562.

Once a button of the universal sensor device is selected for configuration, an example button setup interface 570 is shown in FIG. 5C. An instruction 572 is also displayed that indicates the specific button that has been selected for configuration. As shown in the example in FIG. 5C, button A has been selected. The interface 570 includes a set up medication option 580, a set up dose option 582, a set up a trigger option 584 and a set up a symptom option 586. Each of the options allows the button to be configured to enter different types of data in relation to the general selection as explained above. Once the configuration is complete, the user is returned to the sensor interface 550 in FIG. 5B for configuration of other buttons. After each button is configured with separate interfaces as will be explained below, the interface 550 will show a check mark or other indicator that indicates which buttons have been configured.

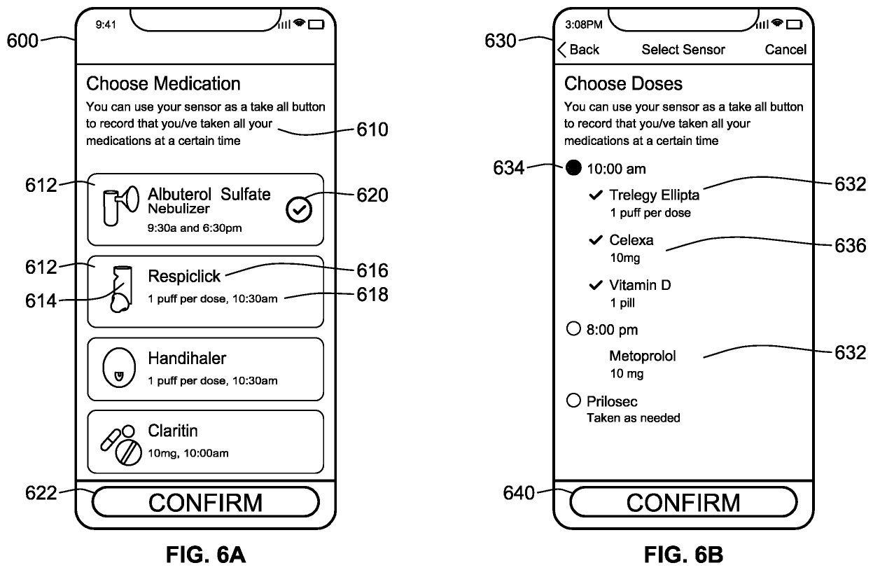

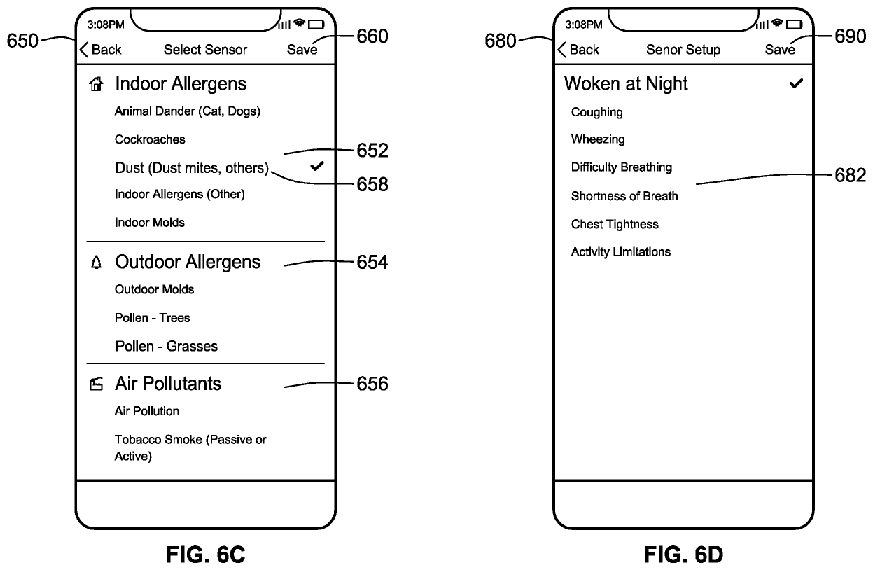

In this example, the set up a medication option 580 has been selected. A medications interface 600 is displayed as shown in FIG. 6A that lists specific medications in a medication field 610. Each of the current medications taken by the user are listed as individual medication selections 612. Each of the selections 612 includes a graphic of the medication 614, a label 616 and the times 618 such medications should be taken. Each of the selections may be selected by the user to be assigned to the button. When the user selects one of the selections 612, a check mark icon 620 appears in the selection 612. A confirmation button 622 is displayed to allow the user to confirm their selection of the assignment to the button. When the confirmation button 622 is selected, the button is assigned to the selected medication and time. Thus, when the configured button is pressed on the universal sensor 100, data is recorded indicating the patient has taken the selected medication.

If the set up a dose option 580 is selected in the button setup interface 570 in FIG. 5C, a dose interface 630 shown in FIG. 6B is displayed. The dose interface 630 lists specific medications grouped by times when they should be taken. Each group such as the group 632 is associated with a specific time represented by a selection bubble 634. Each group lists the medication types and dosages 636 that should be taken at the time of the group. Each of the listings may be selected by the user. A confirmation button 640 is displayed to allow the user to confirm their selection of the assignment of the time group to the button. Thus, when the configured button is pressed on the universal sensor 100, data is recorded indicating the patient has taken the selected medications for the time.

If the set up a trigger option 584 is selected in the button setup interface 570 in FIG. 5C, a trigger option interface 650 shown in FIG. 6C is displayed. In this example, the triggers are grouped into indoor allergens 652, outdoor allergens 654, and air pollutants 656 on the interface 650. Each of the groups 652, 654, and 656 lists specific trigger options such as specific indoor allergen triggers 658. Each of the specific trigger options 658 may be selected by the user and therefore assigned to the button. An icon such as a check mark indicates a specific trigger is selected. A save button 660 allows the user to confirm and save their selection of the assignment to the button. Thus, when the configured button is pressed on the universal sensor 100, data is recorded indicating the patient has experienced the configured trigger.

If the set up a symptom option 586 is selected in the button setup interface 570 in FIG. 5C, a trigger option interface 680 shown in FIG. 6D is displayed. The interface 680 lists specific respiratory based symptoms 682. Each of the specific symptoms listed may be selected by the user. In this example, the symptoms 682 include woken at night, coughing, wheezing, difficulty breathing, shortness of breath, chest tightness and activity limitations. Other symptoms may be provided according to known respiratory ailments that a user may be susceptible to. An icon such as a check mark indicates a specific symptom is selected. A save button 690 is displayed to allow the user to save and confirm their selection of the assignment to the button. Thus, when the configured button is pressed on the universal sensor 100, data is recorded indicating the patient has experienced the configured symptom.

After configuring the buttons of a universal sensor device, a summary screen 590 in FIG. 5D may be displayed that shows the configuration of each button area 592. In this example, each of the buttons has been assigned to a particular medication. The summary screen 590 also displays the last known location of the sensor and the date based on the advertising signals obtained by the smartphone 210. Once such signals are received from a recognized sensor, the application records the time and geographical position of the sensor based on the GPS data acquired by an internal GPS receiver in the smartphone 210.

Thus, when the smartphone synchronizes with a universal sensor, any events that take place within a period of a known location of the smartphone 210 can be associated with that location under the assumption that the smartphone 210 was close to the sensor at the time of the event. In this example, the universal sensor 100 captures the timestamp of the event, and the smartphone 210 associates the location based on its own location at the time of the event. The summary screen 590 also includes a ring sensor input that triggers the specific universal sensor to start emitting an audio signal such as a buzzer or a chime to enable the universal sensor to be located.

Similar to the individual button configuration process, the application may allow a universal sensor 100 paired with the smartphone 210 to be configured for accessibility in relation to a single data entry button such as for the example sensor 380 incorporated with an actuator that allows greater accessibility as shown in FIG. 3D. Thus, similar interfaces are displayed as the interfaces in FIG. 5C to configure an accessibility sensor. In the case of accessibility, the user only configures a single accessibility type button synonymous with the universal sensor such as the universal sensor 380 with options to keep track of doses, medication, activities, symptoms, or emotions.

Returning to FIG. 5A, in this example, the menu displayed after the add icon 506 is selected may include both a configure sensor option (discussed above) as well as a configure medication option. The configure medication option includes interfaces that allow a user to add new medications that after being added are displayed on the medication interface shown in FIG. 5A. The medication entry interfaces allow the selection of available medications, the selection of times to take the medication, icons representing the medications, and delivery devices associated with the medication.

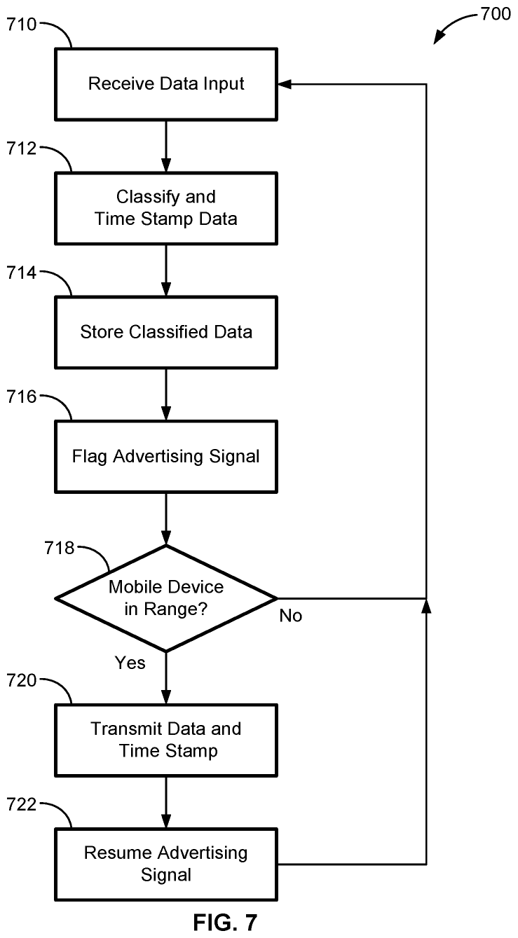

FIG. 7 is a flow diagram 700 of the operational routine executed by the controller 222 of the universal sensor 100 and the application on the remote mobile device 210. The flow diagram 700 in FIG. 7 is representative of example machine readable instructions for collecting and analyzing data collected from the universal sensor device 100 in FIG. 1. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIG. 3, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The controller 222 first determines data has been input from the button interface 224 (710). The controller 222 classifies the data according to the configuration and applies a time stamp (712). For example, if the universal sensor device 100 is configured for collecting data for medications, the controller 222 classifies the collected data as an indication that a medication has been taken by the user at a specific time. The data is then stored in the memory 230 (714). The controller 222 then causes the transceiver 228 to flag the advertising signal emitted by the universal sensor 100 (716). The controller 222 determines whether a paired mobile device is in transmission range (718). If no paired mobile device is in transmission range, the controller 222 continues to collect data (710) and transmit the advertising signal.

If a paired mobile device is within transmission range (718), the controller 222 will send the collected data to the mobile device 210 (720). The mobile device 210 may then transmit the received data to the server 220 for further analysis. The mobile device 210 may transmit the received data at a predefined period of time. In this example, the predefined period of time may be 24 hours. Alternatively, the server 220 may request transmission of a data packet from the mobile device 210 on a periodic basis or when the server 220 determines a medication or other condition is probably present with the patient based on past patterns. The controller 222 of the universal sensor 100 will change the advertising signal to normal (722). The routine then will loop back to collect further data (710).

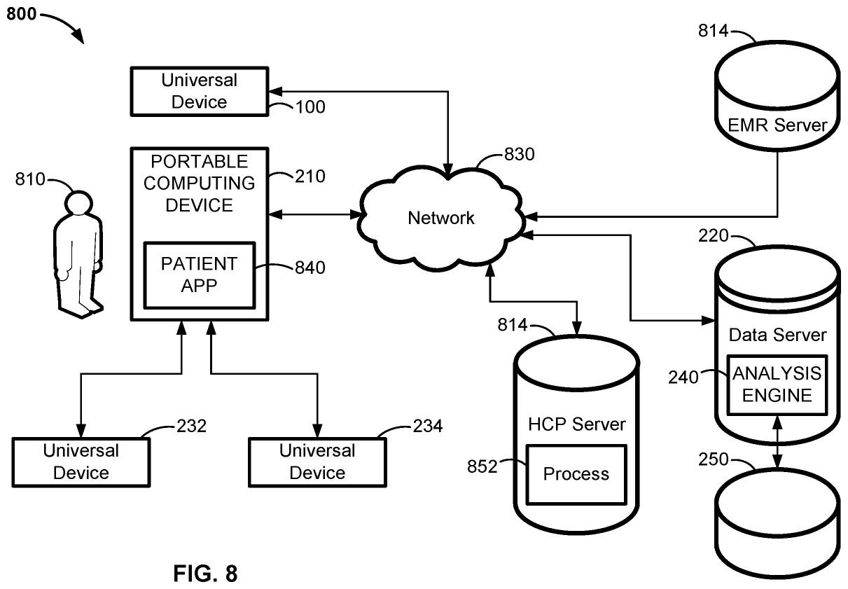

FIG. 8 is a block diagram of an example health care system 800 for obtaining data from patients via universal sensors. The health care system 800 includes a universal sensor the universal sensor 100 in FIG. 1 that provides relevant medication data that is input by a user or patient 810. It is to be understood that there may be multiple universal sensors such as sensors 232 and 234 that are configured for different activities. The universal sensors 100, 232, and 234 may be in different locations that are appropriate for collected the data of the respective configuration. For example, the universal sensor 100 may be configured for medication and therefore be attached to a medicine cabinet. The universal sensor 232 may be configured to record activity and therefore may be attached to the frame of the front door of the dwelling of the patient 810. As explained herein, the universal sensors 100, 232, and 234 are configured by the portable computing device 210 via a patient program or application 840, after pairing the universal sensors 100, 232, and 234 with the computing device 210.

The health care system 800 includes the data server 220, a health or home care provider (HCP) server 814, an electronic medical records (EMR) server 816, and the patient mobile computing device 210. In addition, one or more physiological sensors may provide physiological data from the patient 810. The patient computing device 210 is generally carried on by the patient 810, while the universal sensors 100, 232, and 234 are placed in convenient locations appropriate to the data they are configured to collect. In the system 800, these entities are all connected to, and configured to communicate with each other over, a wide area network 830, such as the Internet. The connections to the wide area network 830 may be wired or wireless. The EMR server 814, the HCP server 816, and the data server 220 may all be implemented on distinct computing devices at separate locations, or any sub-combination of two or more of those entities may be co-implemented on the same computing device.

The patient computing device 210 may be a personal computer, mobile phone, tablet computer, or other device. The patient computing device 210 is configured to intermediate between the patient 810 and the remotely located entities of the system 800 over the wide area network 830. In the implementation of FIG. 8, this intermediation is accomplished by the software application program 840 that runs on the patient computing device 210. In this example, the application program 840 allows the configuration of the data type collected by the universal sensors 100, 232, and 234. The application program 840 also collects the data from the configured universal sensors 100, 232, and 234 when each sensor is within transmission range of the computing device 210.

The patient program 840 may be a dedicated application referred to as a “patient app” or a web browser that interacts with a website provided by the health or home care provider. In this example, the universal sensors 100, 232, and 234 communicate with the patient computing device 210 via a local wired or wireless network (not shown) based on a protocol such as Bluetooth. The system 800 may include other pairings of universal sensors (not shown) associated with respective patients who also have respective associated computing devices and associated HCP servers (possibly shared with other patients). All the patients/users in the system 800 may be managed by the data server 220.

As explained above, the data from the universal sensor 100 configured to collect medication uses may be correlated with the application of drug doses by the patient 810. Additional data from the universal sensors 232 and 234 may be collected by the computing device 210 to track the effectiveness of the drug in applying the drug as explained above in relation to the health data analysis module 240. Such data is collected by the application 840 and sent to the data server 220. In this example, the analysis module 240 may provide analysis of the collected data such as determining effectiveness of the drug, tracking dosages taken by individual patients such as the patient 810, or identifying patterns of behavior and environmental conditions with dosages. If the universal sensors 100, 232, and 234 are collecting different types of data, different analysis relating to the patient 810 may be performed.

In this example, the universal sensor 100 is configured to transmit inputs indicating taking a specific medication to the patient computing device 210 via a wireless protocol, which receives the data as part of the patient program 840. The patient computing device 210 then transmits the physiological data to the data server 220 according to pull or push model. The data server 220 may receive the physiological data from the computing device 210 according to a “pull” model whereby the computing device 210 transmits the medication data in to a query from the data server 220. Alternatively, the data server 220 may receive the physiological data according to a “push” model whereby the computing device 210 transmits the data to the data server 220 as soon as it is available after an input is detected from the patient. The data server 220 may access databases such as the database 250 to store collected and analyzed data.

Data received from the patient computing device 210 is stored and indexed by the data server 220 so as to be uniquely associated with patient 810 and therefore distinguishable from physiological data collected from any other universal sensors in the system 800. The data server 220 may be configured to calculate summary data for each dose application from the data received from the universal sensor 100. The data server 220 may also be configured to receive data from the patient computing device 210 including other data entered by the patient 810, behavioral data about the patient, or dose/summary data.

The EMR server 814 contains electronic medical records (EMRs), both specific to the patient 810 and generic to a larger population of patients with similar disorders to the patient 810. An EMR, sometimes referred to as an electronic health record (EHR), typically contains a medical history of a patient including previous conditions, treatments, co-morbidities, and current status. The EMR server 814 may be located, for example, at a hospital where the patient 810 has previously received treatment. The EMR server 814 is configured to transmit EMR data to the data server 220, possibly in response to a query received from the data server 220.

In this example, the HCP server 816 is associated with the health/home care provider (which may be an individual health care professional or an organization) that is responsible for the patient's respiratory therapy. An HCP may also be referred to as a DME or HME (domestic/home medical equipment provider). The HCP server 816 may host a process 852 that is described in more detail below. One function of the HCP server process 852 is to transmit data relating to the patient 810 to the data server 220, possibly in response to a query received from the data server 220.

In some implementations, the data server 220 is configured to communicate with the HCP server 816 to trigger notifications or action recommendations to an agent of the HCP such as a nurse, or to support reporting of various kinds. Details of actions carried out are stored by the data server 220 as part of the engagement data. The HCP server 816 hosts an HCP server process 852 that communicates with the analysis module 240 and the patient program 840.

For example, the HCP server process 852 may include the ability to monitor the patient taking certain medication in accordance with compliance rules that specify the required medication usage over a compliance period, such as 30 days, in terms of a minimum number of doses, such as four times, for some minimum number of days, e.g. 21 days, within the compliance period. The summary data post-processing may determine whether the most recent time period is a compliant session by comparing the usage time with the minimum duration from the compliance rule. The results of such post-processing are referred to as “compliance data.” Such compliance data may be used by a health care provider to tailor therapy that may include the inhaler and other mechanisms. Other actors such as payors may use the compliance data to determine whether reimbursement may be made to a patient. The HCP server process 852 may have other health care functions such as determining overall use of drugs based on collection of data from numerous patients. For example, health care organizations may run analysis algorithms to determine correlations between populations of patients using their drugs effectively and reduced hospitalization rates. Another example is determination of physiological patterns of pre- and post-drug use that indicate if a is at risk of imminent rehospitalization. Another example is an analysis of the effectiveness of different drug brands (but same drug type) in managing patient health.

As may be appreciated data in the data server 220, EMR server 814 and HCP server 816 is generally confidential data in relation to the patient 810. Typically, the patient 810 must provide permission to send the confidential data to another party. Such permissions may be required to transfer data between the servers 220, 814 and 816 if such servers are operated by different entities.

The analysis module 240 may collect the configured data from the universal sensors 100, 232, and 234 through the patient computing device 210. The analysis module 240 generally provides analysis on treatment of the patients based on the collected data. For example, data from dosage application and the corresponding data such as triggers and symptoms from other universal sensors and may be used to determine a customized dosage for the individual user. Other stored data may be used to further tailor a dosage. The customized dosage may be therefore determined based on the physiology, disease type and activity specific to the user. The additional data may be stored in the database 250 and be used by the analysis module 240 to assist in personalized analysis of the data received from the use of the configured universal sensor 100. Other analysis may be performed. For example, if a pattern is identified with pollen or other environmental triggers from the collected data, then external weather data could be used to notify the patient of the likelihood of a needed dosage. A pattern of a particular activity followed by a medication usage may trigger intervention if the pattern is broken. Thus, if the patient always takes a dosage after they go for a run every Tuesday, and they log a run Tuesday but with no medication usage, then the system may send a notification to the patient reminding them. If patient-reported sleep quality is steadily declining based on the inputs collected by a universal sensor, an intervention by a physician can be triggered. If the user fails to perform a specific scheduled activity (such as exercise) for a period as determined from the collected data, the analysis may determine that the patient is subject to an impending exacerbation or a general decline of wellbeing.

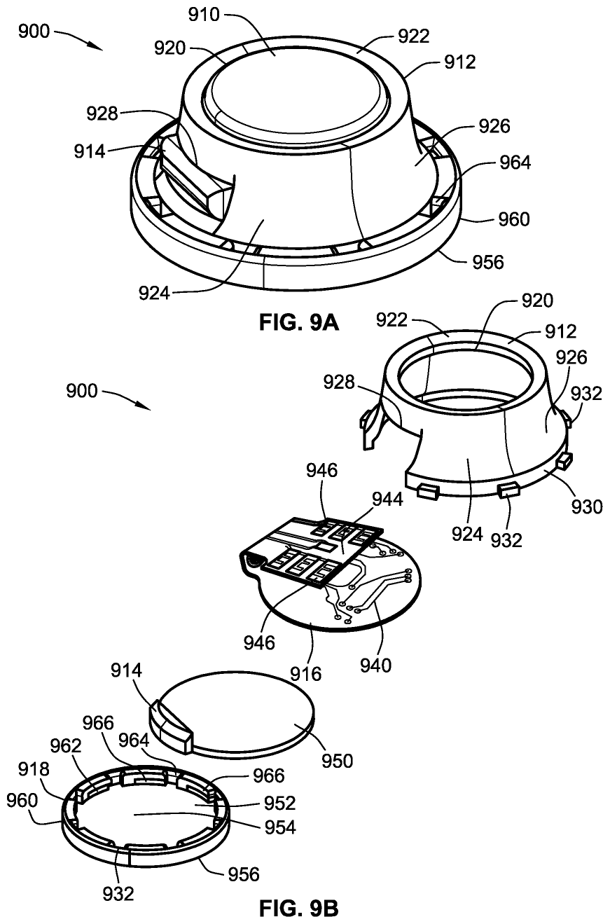

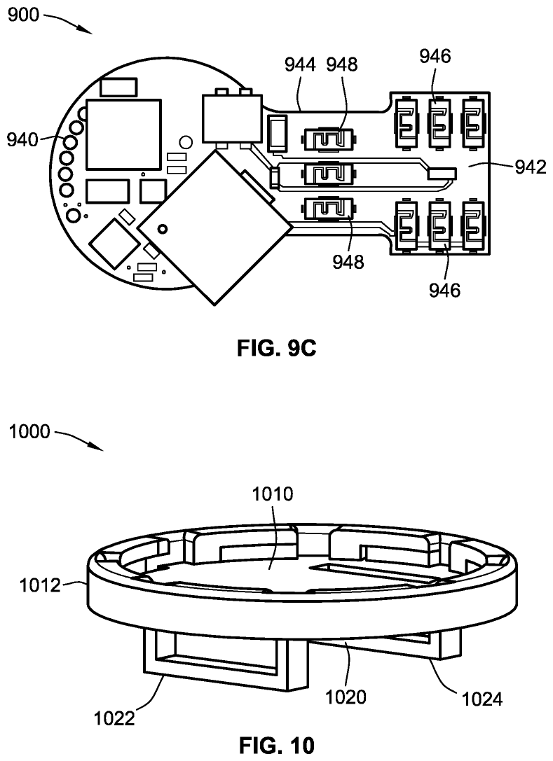

FIG. 9A is a perspective view of another example universal sensor device 900 that has a single programmable button 910. FIG. 9B is an exploded view of the components of the universal sensor device 900. As shown in FIG. 9B, the universal sensor 900 includes a main conical body component 912, an auxiliary button 914, a flexible circuit board 916 and a base plate member 918. The conical body component 912 has an open circular top aperture 920 defined by an annular top collar 922. The top aperture 920 holds the programmable button 910. In this example the programmable button 910 has a cylindrical body that holds the button 910. The button 910 has a top surface that may be pressed by a user. The button 910 is a silicone molded piece in this example, but other flexible materials such as rubber may be used. An opposite surface may be coated with a conductive material such as carbon or silver paint. Thus, when the button 910 is pressed, the conductive coating makes contact with traces on the underlying circuit board 916 and closes the circuit. Alternatively, the circuit board 916 may have open traces and a metal snap dome. When the button is pressed, the metal on the snap dome is deformed and makes a connection between two contacts on the circuit board 916. A decal or other visual indicator may be applied to the top surface of the button 910 to indicate the configured function such as recording a certain type of medication when taken by the user.

The top collar 922 is defined by two semi-circular side panels 924 and 926. One of the side panels 924 includes a side aperture 928 for access the auxiliary button 914. The bottom of the side panels 924 and 926 define a bottom annular collar 930. The bottom annular collar 930 has a series of periodically spaced tabs 932. In this example, the side panels 924 and 926 are fabricated from rigid plastic.

As shown in FIG. 9C, the flexible circuit board 916 is manufactured as a single plane board. The flexible circuit board 916 includes a circular main section 940 and a rectangular section 942 that is joined via a rectangular arm section 944. In this example, the material is polyimide that may be imprinted with conductor patterns for electrically connecting the components on the circuit board 916. The polyimide material allows the circuit board 916 to be folded over. As shown in FIG. 9B, the rectangular section 942 is folded over to a parallel position relative to the circular main section 940 to enclose the battery. Thus, the battery provides power through contacts on the rectangular section 942 and the main section 940. The battery also serves to support the rectangular section 942.

The main section 940 includes a transceiver circuit, signal processing circuits, driver circuits, and a controller. The electronic components in the main section 940 are similar to the electronic components of the universal sensor 100 shown in FIG. 2. Thus, the electronic components will record button pushes and correlated programmed data collection functions based on the recorded pushes as explained previously. The transceiver may be paired with a mobile device as explained above in reference to FIG. 2 for collection of the data as well as programming the type of data collected by the button pushes.

The rectangular section 942 includes six finger contacts 946. The contacts 946 are fixed in a horizontal position via the rectangular section 942 being supported by the battery. Any of the contacts 946 close a circuit when the button 910 is pushed. The output of the completed circuit of the contacts 946 is thus processed by the signal processing circuitry and converted to digital data representing a button push that is managed by the controller. The arm section 944 also has three finger contacts 948 that may complete a circuit when the auxiliary button 914 is pushed.

The auxiliary button 914 is mounted on the edge of a disc-shaped platform 950. The auxiliary button 914 may be moved relative to the platform 950 to contact the finger contacts 948. The platform 950 supports the main section 940 of the circuit board 916. The platform 950 and the circuit board 916 thus are enclosed by the conical body component 912. In this example, the auxiliary button 914 has pre-determined functions that may be activated when the auxiliary button 914 is pushed and completes a circuit through contact with the finger contacts 948. Example pre-determined functions include turning on and off an alarm or triggering a manual sync, that cannot be configured by a user. Thus, the auxiliary button 914 is user operated, but activates a non-configurable function, that is set by the controller.

The base plate member 918 has a circular bottom panel 952. An interior upper surface 954 of the bottom panel 952 supports the platform 950. An opposite exterior lower surface 956 of the circular bottom panel 952 may be coated with an adhesive to allow the sensor 900 to be attached to a flat surface. In this example, the adhesive may be covered by a backing layer that may be removed when a user is ready to attach the sensor 900 to a flat surface.

The base plate member 918 has an annular wall 960 that includes an interior surface 962. A series of vertical slots 964 and horizontal slots 966 are formed on the interior surface 962. The universal sensor 900 is assembled by seating the platform 950 on the upper surface 954 of the base plate member 918. The circuit board 916 is seated on the platform 950 and the battery is inserted. The circuit board 916 is then folded over as shown in FIG. 9B. The body component 912 is then lowered such that the tabs 932 are inserted into each of the corresponding vertical slots 964. The body component 912 is then rotated in counter-clockwise direction so the tabs 932 move to one end of the corresponding horizontal slots 966. The body component 912 may be removed from the base plate member 918 by rotating it in a clockwise direction to allow the tabs 932 to move to the opposite end of the corresponding horizontal slots 966.

As explained above, the universal sensor 900 may be paired with a mobile device such as the smartphone 210 in FIG. 2. The controller of the universal sensor 900 may be programmed by the application on the smartphone 210 to associate actions with the push of the button 910. Thus, the button 910 is an example of a user operated configurable input. The configurations are described above and may include recording medication usages.

In this example, the base plate member 918 of the universal sensor 900 may be attached to any convenient flat surface. However, there may be other ways to attach the universal sensor 900. FIG. 10 shows an alternate base plate member 1000 for the universal sensor 900. The base plate member 1000 includes a circular bottom panel 1010 with an annular wall 1012 with horizontal and vertical slots that allow the mating of the body component 912 in FIG. 9A. The circular bottom panel 1010 has a bottom exterior surface 1020 that has two strap holders 1022 and 1024. A strap, elastic band, or lanyard may be inserted through the strap holders 1022 and 1024 to be looped around an object such as a pole or the wrist of a user. The strap or lanyard may have an adjustable length with an attachment mechanism such as Velcro or the like.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1-59 below can be combined with one or more elements or aspects or steps, or any portion(s) thereof, from one or more of any of the other claims 1-59 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A configurable sensor directed toward collection of user input data, the sensor comprising:
a user operated configurable input;
a transceiver operable to communicate with an external device; and
a controller coupled to the configurable input and the transceiver, the controller operable to collect data from the configurable input and classify the data according to a configured data type.

2. The sensor of claim 1, wherein the configured data type is one of user medication, user dose, user emotion, user activity, or user trigger.

3. (canceled)

4. The sensor of claim 1, further comprising a housing holding the transceiver and controller, the housing having a base exterior surface and an opposite open aperture, wherein the user operated configurable input is a push-button extending through the open aperture.

5. The sensor of claim 4, wherein the base exterior surface includes one of an adhesive for attachment to a flat surface or a loop structure for attaching a lanyard or strap.

6. (canceled)

7. The sensor of claim 1, further comprising a faceplate having a plurality of button areas, wherein the configurable input is one of the plurality of button areas.

8. (canceled)

9. The sensor of claim 7, further comprising a decal overlaying the faceplate, wherein the decal includes identification information for the button areas associated with the configured data type, and wherein the decal includes icons on each of the button areas, wherein the icons correspond to the configured data type.

10. (canceled)

11. The sensor of claim 7, wherein the configured data type includes a plurality of related data inputs, each data input corresponding to each one of the plurality of button areas.

12-13. (canceled)

14. The sensor of claim 1, wherein the external device is a mobile device, wherein the controller sends the collected data via the transceiver to the mobile device when the mobile device is within transmission range of the configurable sensor.

15. (canceled)

16. The sensor of claim 14, wherein the mobile device is paired with the configurable sensor for collecting the collected data, and wherein the mobile device is paired with a second configurable sensor comprising:
a user operated configurable input;
a transceiver operable to communicate with an external device; and
a controller coupled to the configurable input and the transceiver, the controller operable to collect data from the configurable input and classify the data according to a configured data type, wherein the controller sends the collected data via the transceiver to the mobile device when the mobile device is within transmission range of the second configurable sensor.

17. (canceled)

18. The sensor of claim 14, wherein the mobile device includes a configuration interface operable to configure the configured data type of the configurable sensor corresponding to the configurable input, wherein the mobile device is operable to send a configuration signal to the transceiver to configure the configurable input and the configurable data type.

19. The sensor of claim 1, wherein the user operated input is an actuator device coupled remotely from the transceiver and the controller.

20. (canceled)

21. A data collection system, the system comprising:
a first configurable sensor directed toward collection of user input data, the sensor including:
a user operated configurable input;
a transceiver operable to communicate with an external device; and
a controller coupled to the configurable input and the transceiver, the controller operable to collect data from the configurable input and classify the data according to a configured data type;
a data server to receive the collected classified data from the transceiver; and
an analysis module operable to determine a health condition of the user based on the collected classified data.

22. The data collection system of claim 21, wherein the configured data type is one of user medication, user dose, user emotion, user activity, or user trigger.

23-32. (canceled)

33. The data collection system of claim 21 further comprising a mobile device, wherein the controller sends the collected data via the transceiver to the mobile device when the mobile device is within transmission range of the configurable sensor and wherein the mobile device sends the collected data to the data server.

34-36. (canceled)

37. The data collection system of claim 33, wherein the mobile device includes a configuration interface operable to configure the configured data type of the configurable sensor corresponding to the configurable input, wherein the mobile device is operable to send a configuration signal to the transceiver to configure the configurable input and the configurable data type.

38. (canceled)

39. A method of collecting user input data from a universal sensor, the method comprising:
configuring a user operated configurable input on the universal sensor according to a configured data type;
receiving an input on the user operated configurable input;
collecting the data from the configurable input via a controller;
classifying the data according to the configured data type; and
transmitting the classified data via a transceiver to an external device.

40. The method of claim 39, wherein the configured data type is one of user medication, user dose, user emotion, user activity, or user trigger.

41-50. (canceled)

51. The method of claim 39, wherein the external device is a mobile device, wherein the controller sends the collected data via the transceiver to the mobile device when the mobile device is within transmission range of the configurable sensor.

52. (canceled)

53. The method of claim 51, further comprising:
pairing the mobile device with the configurable sensor; and
pairing the mobile device with a second configurable sensor, wherein the second configurable sensor includes:
a user operated configurable input;
a transceiver operable to communicate with an external device; and
a controller coupled to the configurable input and the transceiver;
collecting data from the configurable input of the second configurable sensor;
classifying the data according to a configured data type; and
sending the collected data via the transceiver to the mobile device when the mobile device is within transmission range of the second configurable sensor.

54. (canceled)

55. The method of claim 51, further comprising:
configuring the configured data type of the configurable sensor corresponding to the configurable input via a configuration interface of the mobile device; and
sending a configuration signal from the mobile device to the transceiver to configure the configurable input and the configurable data type.

56-59. (canceled)
